(19) United States
(12) Patent Application Publication
(10) Pub. No.: US 2011/0059431 A1
(43) Pub. Date: Mar. 10, 2011

(54) NON-ENZYMATIC DETECTION OF BACTERIAL GENOMIC DNA

(75) Inventors: Chad A. Mirkin, Wilmette, IL (US); Rafael A. Vega, Evanston, IL (US); Haley D. Hill, Evanston, IL (US)

(73) Assignee: NORTHWESTERN UNIVERSITY, Evanston, IL (US)

(21) Appl. No.: 12/665,326

(22) PCT Filed: Jun. 18, 2008
(86) PCT No.: PCT/US08/67417

§ 371 Date: Nov. 19, 2010

Related U.S. Application Data
(60) Provisional application No. 60/944,676, filed on Jun. 18, 2007.
(60) Provisional application No. 60/936,957, filed on Jun. 22, 2007.

(51) Int. Cl. C12Q 1/70; C12Q 1/68

(52) U.S. Cl. 435/5; 435/6

(57) ABSTRACT

The present invention relates to methods for detecting for the presence of one or more target analytes, e.g. biomolecules, in a sample. In particular, the present invention relates to a method that utilizes blocking strands to inhibit target rehybridization to detect double stranded genomic DNA.

### CROSS-REFERENCE TO RELATED APPLICATIONS

This application claims priority of U.S. Provisional Application No. 60/944,676 filed Jun. 18, 2007, and U.S. Provisional Application No. 60/936,957 filed Jun. 22, 2007, both of which are hereby incorporated by reference in its entirety.

### STATEMENT OF GOVERNMENT INTEREST

This invention was made with Government support under grant number EEC-0647560 awarded by The National Science Foundation (NSF)/Nanoscale Science and Engineering Centers (NSEC), and grant number F49620-01-1-0401, awarded by The Air Force Office of Scientific Research (AFOSR). The Government has certain rights in the invention.

### FIELD OF THE INVENTION

The present invention concerns a method of detecting genomic DNA using nanoparticles functionalized with binding agents. More specifically, the invention provides a diagnostic assay for the detection of genomic DNA utilizing a modification of the biobarcode assay wherein blocking polynucleotides (blockers) are used to prevent rehybridization of the genomic DNA.

### BACKGROUND OF THE INVENTION

Polymerase chain reaction (PCR)-based amplification techniques (Saiki et al., Science 230: 1350-1354 (1985); Mullis et al., Cold Spring Harbor Symposia on Quantitative Biology 51: 263-273 (1986); Scharf et al., Science 233: 1076-1078 (1986)) have become standard methodologies for the detection of nucleic acids (Kary, Angewandte Chemie International Edition in English 33: 1209-1213 (1994); Jochen Wilhelm, ChemBioChem 4: 1120-1128 (2003)). With the advent of quantitative real time. PCR and variants of it such as reverse transcription PCR, one can now detect nucleic acid targets in a highly quantitative manner and assess important processes like gene expression (Bowtell, DNA microarrays: a molecular cloning manual; Cold Spring Harbor Press: Cold Spring Harbor, N.Y. (2003); DeRisi et al., Science 278: 680-686 (1997); Gibson et al., Genome Research 6: 995-1001 (1996); Heid et al., Genome Research 6: 986-994 (1996); Higuchi et al., Bio-Technology 11: 1026-1030 (1993)). Though there are many benefits to PCR such as sensitivity, production of a usable product fragment, and the ability to sequence that fragment, there are times when these features of PCR are not necessary and the cumbersome nature of PCR is a disadvantage. For example, in the case of point-of-care biological detection applications, where speed is critical and the enzymatic constraints and cost of PCR are limiting (Mirkin et al., Expert Review of Molecular Diagnostics 4: 749-751 (2004)), an enzyme-free approach could be a major advantage.

The recently developed bio-barcode assay (see, for example, U.S. Pat. No. 6,974,669 and U.S. Pat. No. 7,323,309, each of which is hereby incorporated by reference in its entirety) for the detection of protein and nucleic acid targets is potentially capable of filling this void. This assay has several forms (Nam et al., Journal of the American Chemical Society 124: 3820-3821 (2002); Nam et al., Journal of the American Chemical Society 126: 5932-5933 (2004); Stoeva et al., Angewandte Chemie International Edition 45: 3303-3306 (2006); Stoeva et al., J. Am. Chem. Soc. 128: 8378-8379 (2006); Thaxton et al., Analytical Chemistry 77: 8174-8178 (2005); Georganopoulou et al., Proceedings of the National Academy of Sciences of the United States of America 102: 2273-2276 (2005)), and has shown promise in the high sensitivity detection of single protein and polynucleotide targets. In addition, it has the ability to simultaneously detect many different targets in one sample.

Gold nanoparticles functionalized with polynucleotides (oligo-AuNPs), are the cornerstone of the bio-barcode assay (Mirkin et al., Nature 382: 607-609 (1996)). These oligo-AuNPs have a variety of attributes with respect to probe design. They are easily functionalized (Mirkin et al., Nature 382: 607-609 (1996)), highly tailorable (Li et al., Nucleic Acids Research 30: 1558-1562 (2002); Cao et al., Science 297: 1536-1540 (2002)), remarkably stable (Storhoff et al., Journal of the American Chemical Society 122: 4640-4650 (2000)), catalytic (Taton et al., Science 289: 1757-1760 (2000)), and cooperative binders (they exhibit unusually sharp melting transitions when hybridized to complementary DNA). These sharp melting transitions can confer a considerable selectivity advantage to the oligo-AuNPs over their PCR primer counterparts (Lytton-Jean et al., Journal of the American Chemical Society 127: 12754-12755 (2005)). Oligo-AuNPs serving as amplification agents in the bio-barcode assay, through the chemical release of their polynucleotide “barcodes,” have several potential advantages over Taq-Polymerase or other DNA replication enzymes. For example, the oligo-AuNP probes are stable for extended periods (greater than 6 months) at ambient temperature (Storhoff et al., Chemical Reviews 99: 1849-186226 (1999)), while polymerases, like most enzymes, need to be stored at 4° C. Oligo-AuNPs also function in a host of complex conditions such as sodium chloride concentrations up to 1 M, different buffers such as Tris, Phosphate, Borate, Mops and in the presence of metal ions or small molecules without adverse effect to their activity (Han et al., Angewandte Chemie-International Edition 45: 1807-1810 (2006); Han et al., Journal of the American Chemical Society 128: 4954-4955 (2006); Lee et al., Angewandte Chemie International Edition 46: 4093-4096 (2007)).

The bio-barcode assay combines a first homogenous capture agent (a magnetic microparticle functionalized with a different target specific polynucleotide, oligo-MMP, or alternatively, any solid surface that promotes and/or allows separation is herein referred to as a “first particle”) with a second target specific oligo-AuNPs (herein referred to as a “second particle”). The oligo-MMP is used to capture and isolate the target of interest from a complex and/or dirty sample solution, prior to the addition of the oligo-AuNPs. The MMP-target-AuNP complex allow for rapid isolation and subsequent washing prior to polynucleotide barcode release. The barcodes can be easily detected via the scanometric method, which exhibits an LOD for short purified polynucleotides of 100 aM (Taton et al., Science 289: 1757-1760 (2000); Storhoff et al., In Microarray Technology and Its Applications (2005); Mueller, U. R. N., Dan V., Ed.; Springer GmbH: Berlin, Germany, pp 147-179). Recently it has been adapted to a microfluidic chip-based format, an important step towards automation (Goluch et al., Lab on a Chip 6: 1293-1299 (2006)). With respect to nucleic acids, thus far all proof-of-concept work has involved short nucleic acids in very clean environments (i.e., buffer). The complexities of the target and sample media are often limiting factors for any nucleic acid assay, especially ones that rely on enzymes for amplification.

There thus remains an unmet need in the art for a method of detecting double stranded DNA in a sample without the need to rely on enzymes for amplification.

### SUMMARY OF THE INVENTION

The bio-barcode assay can overcome limitations currently associated with detection of target double stranded DNA. Herein, the development of a new version of the bio-barcode assay is disclosed that utilizes blocking strands to inhibit target rehybridization and allows one to detect double stranded genomic DNA at a limit-of-detection (LOD) of 2.5 fM. Proof-of-concept studies in the context of Bacillus subtilis DNA are exemplified.

In one embodiment, a method for detecting presence of a target polynucleotide in a sample is provided comprising the step of detecting the target polynucleotide in a particle complex, components of the particle complex comprising the target polynucleotide a first particle having a first polynucleotide attached thereto, wherein all or part of the first polynucleotide is specifically hybridized to a first binding complement in the target polynucleotide a second particle having a second polynucleotide attached thereto and a DNA barcode hybridized to a first site in said second polynucleotide, wherein the second polynucleotide is specifically hybridized to a second binding complement in the target polynucleotide through a second site in said second polynucleotide, and a blocking polynucleotide hybridized to a third binding complement in the target polynucleotide, wherein hybridization of the blocking polynucleotide to the target polynucleotide prevents the target polynucleotide from hybridizing to its complementary sequence, wherein the particle complex is in an environment that promotes dehybridization of the DNA barcode from the complex, the detection of the DNA barcode indicating the presence of the target polynucleotide.

In one aspect, the particle complex is isolated prior to dehybridization of the DNA barcode.

In another aspect, the particle complex is formed by sequential addition of one or more solutions of components which form the particle complex to a solution containing the target polynucleotide.

In still another aspect, the particle complex is formed by sequential addition of a solution containing the target polynucleotide to one or more solutions of components which form the particle complex.

In some embodiments, the first particle is magnetic.

In other embodiments, the particle complex is isolated using a magnet prior to dehybridization of the DNA barcode.

In an aspect of the methods, the second particle is a nanoparticle.

In an embodiment, the nanoparticle is a metallic nanoparticle.

In some aspects, the metallic nanoparticle is a gold nanoparticle.

In another aspect, the target is a naturally occurring polynucleotide.

In still another aspect, the target polynucleotide is a synthetic polynucleotide.

In another aspect, the synthetic polynucleotide is a peptide nucleic acid.

In an embodiment, the target polynucleotide is a polynucleotide that forms intermolcular or intramolecular double-stranded structure that precludes particle complex formation.

In some aspects, the molecule is selected from the group consisting of DNA or RNA.

In an embodiment of the methods, the target polynucleotide is a bacterial polynucleotide.

In another embodiment, the target polynucleotide is bacterial genomic DNA.

In yet another embodiment, the target polynucleotide is a viral polynucleotide.

In some embodiments, the viral polynucleotide is viral genomic DNA.

In an aspect of the methods, the polynucleotide is a fungal polynucleotide.

In another aspect, the fungal polynucleotide is fungal genomic DNA.

In one embodiment, a method is provided for detecting presence of a target polynucleotide in a sample further comprising the steps of denaturing a target polynucleotide having a double stranded polynucleotide region, hybridizing the target polynucleotide to the blocking polynucleotide, hybridizing the target polynucleotide to the first polynucleotide bound to the first particle, washing the target polynucleotide to remove any first polynucleotide on the first particle that is not hybridized to the target polynucleotide, hybridizing the target polynucleotide to the second polynucleotide bound to the second particle, washing the target polynucleotide to remove any second polynucleotide on the second particle that is not hybridized to the target polynucleotide, isolating the particle complex comprising the target polynucleotide having the blocking polynucleotide hybridized thereto, the first polynucleotide on the first particle hybridized thereto, and the second polynucleotide on the second particle hybridized thereto, dehybridizing the DNA barcode from the second polynucleotide bound to the second particle, and detecting the DNA barcode, thereby indicating presence of the target polynucleotide.

The present invention demonstrates the bio-barcode assay's ability to detect DNA down to at least 2.5 fM, with a linear range spanning three orders of magnitude. The integration of blocking polynucleotides proved to be a critical addition to the original bio-barcode method, ultimately allowing for the detection of complex duplex DNA isolated from B. subtilis cells. This work paves the way for the transition of the bio-barcode assay from a laboratory technique to one that can be deployed in the field for the rapid and accurate detection of biological terrorism agents. Thus the present invention contemplates that the bio-barcode assay may be coupled with automated field-deployable sample collection technologies to produce a system for continuous biological surveillance, much like the current Bio-Watch program (Shea et al., Congressional Research Service Report No. RL 32152 (2003)).

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### DESCRIPTION OF THE DRAWINGS

FIG. 1 is a graphical depiction of the genomic bio-barcode assay.

FIG. 2 depicts probe melting analysis. (A) Melting curve for the duplex formed between an oligo-AuNP probe and its fluorophore-labeled complement (sequences given in Table 1). (B) Melting curve for the duplex formed between the oligo-MMP probe and its fluorophore-labeled complement (sequences given in Table 1). The fluorescence of the complementary strands is quenched when they are bound to the AuNP and is recovered when the duplexes melt with the fluorophore strand being released into solution.

FIG. 3 depicts blocking oligonucleotide functionality. (A) Scheme showing how the blocking oligonucleotides are designed to prevent genomic DNA strand rehybridization. (B) This graph shows the importance of the blocking oligonucleotides to the function of the bio bar code assay. It is clearly seen that without blockers the signal obtained in the assay is the same as that with no target, while in the presence of blockers a large signal is obtained indicating that the genomic DNA is available for hybridization to probes.

FIG. 4 depicts the average of five independent runs of the genomic DNA bio-barcode assay and shows that the assay is sensitive down to 2.5 fM target concentration.

### DETAILED DESCRIPTION OF THE INVENTION

The bio-barcode assay has been previously reported, and its uses detailed. See for example U.S. Pat. No. 6,974,669 and U.S. Pat. No. 7,323,309. The present invention is an improvement of the assay, the improvement comprising the use of blocking polynucleotide strands to prevent the rehybridization of double stranded target DNA thereby increasing the number of target strands available for detection by a functionalized particle. The effect of preventing the rehybridization results in an approximate 6-fold increase in signal intensity versus absence of blocking strands.

In various aspects of the methods, the effect of preventing rehybridization of double stranded target DNA results in an increase in signal intensity versus absence of blocking strands of at least 50%, at least 60%, at least 70%, at least 75%, at least 80%, at least 85%, at least 90%, at least 95%, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold or more.

As used herein, the term “blocking strand” or “blocker” means a molecule that is able to hybridize to a portion of a single stranded target polynucleotide sequence under conditions sufficient for hybridization, wherein the hybridization prevents the rehybridization of double stranded target DNA. Although a blocking strand is exemplified herein as being comprised of DNA, one of ordinary skill in the art will recognize that the blocking strand may be comprised of, e.g., any polynucleotide, as well as small molecules, polypeptides or any binding agent(s) which specifically recognize and bind to a target polynucleotide in a manner that prevents hybridization of the double stranded target. Exemplary polypeptides include, but are not limited to target sequence-specific antibodies. Indeed, it is within the skill of those in the art to recognize that any molecule that is capable of binding under conditions sufficient for specific binding to a target polynucleotide sequence wherein its binding prevents the rehybridization of double stranded target DNA, and wherein its binding does not prevent hybridization of a polynucleotide attached to a first or second particle of the invention is contemplated by the invention. These include but are not limited to the various forms of polynucleotides discussed herein below.

As used herein, the term “particle complex” comprises a target polynucleotide, a first particle having a first polynucleotide attached thereto, wherein all or part of the first polynucleotide is specifically hybridized to a first binding complement in the target polynucleotide a second particle having a second polynucleotide attached thereto and a DNA barcode hybridized to a first site in the second polynucleotide, wherein the second polynucleotide is specifically hybridized to a second binding complement in the target polynucleotide through a second site in the second polynucleotide, and a blocking polynucleotide hybridized to a third binding complement in the target polynucleotide, wherein hybridization of the blocking polynucleotide to the target polynucleotide prevents the target polynucleotide from hybridizing to its complementary sequence.

As used herein, the term “target polynucleotide” refers to the single strand of a double stranded DNA to which a polynucleotide attached to either a first or second particle of the invention can hybridize. As used herein, the term “non-target polynucleotide” refers to the strand of a double stranded DNA to which the target polynucleotide can hybridize.

The terms “dehybridizes” or “dehybridizing” is understood in the art to mean a specific dissociation reaction wherein hybridized polynucleotides dissociate or melts, generally brought about by changes in local environmental conditions. In one aspect, the local change is an increase in temperature above a “melting (or dehybridizing) temperature, Tₘ” at which two specific nucleic acids that are hybridized are dissociated by 50%. Changes in local environmental conditions can alter the Tₘ for any given hybridized nucleic acids. While the terms “dehybridizes” or “dehybridizing” is used herein to describe dissociation of hybridized nucleic acids, it will readily be appreciated that dissociation of the interaction between any two other types of binding pair molecules is referred to simply as “dissociation” and this dissociation is, like dehybridizing, affected by local environmental conditions at the site of binding between the binding pair.

The dehybridizing properties of nanoparticle-polynucleotide aggregates are affected by a number of factors, including polynucleotide surface density, nanoparticle size, interparticle distance, and salt concentration. As with native DNA, the Tₘ of these polynucleotide-linked nanoparticle structures increases with increasing salt concentration. However, changes in salt concentration do not substantially affect the sharpness of the transition. The sharp salt-induced melting of the nanoparticle-polynucleotide system, which is not observed in unmodified polynucleotides of the same sequence, allows one to readily discriminate between perfectly complementary targets and single-base mismatched strands and, thus, to develop high selectivity detection assays and potentially eliminate the need for thermal stringency. There also is a strong dependence of Tₘ on interparticle distance; in general, Tₘ increases with increasing interparticle distance for the DNA-linked nanoparticle aggregates due to less electrostatic/steric repulsion and hence stabilization of the duplex interconnects (Jin et al., J. Am. Chem. Soc. 125: 1643-1654 (2003)).

As used herein, “stable” means that, for a period of at least six months after the conjugates are made, a majority of the polynucleotides remain attached to the nanoparticles and the polynucleotides are able to hybridize with nucleic acid and polynucleotide targets under standard conditions encountered in methods of detecting nucleic acid and methods of nanofabrication.

It is to be noted that the term “a” or “an” entity refers to one or more of that entity. For example, “a characteristic” refers to one or more characteristics or at least one characteristic. As such, the terms “a” (or “an”), “one or more” and “at least one” are used interchangeably herein. It is also to be noted that the terms “comprising”, “including”, and “having” have been used interchangeably.

In various aspects of the methods, multiple blocking strands are utilized. In some aspects, three blocking strands may be utilized according to the methods of the invention. The three blocking strands (herein referred to as “5′ blocking”, “3′ blocking” and “center blocking” strands) are complementary and hybridize under appropriate conditions to portions of the target polynucleotide other than those that are complementary to a polynucleotide attached to either the first or second particle of the invention. A 5′ blocking strand will hybridize to a portion of the target polynucleotide that resides on the 5′ (i.e., left) side of a sequence complementary to a polynucleotide sequence attached to a first particle of the invention. A center blocking strand will hybridize to a portion of the target polynucleotide that lies between a sequence complementary to a polynucleotide sequence attached to a first particle of the invention and a sequence complementary to a polynucleotide sequence attached to a second particle of the invention. A 3′ blocking strand will hybridize to a portion of the target polynucleotide that resides on the 3′ (i.e., right) side of a sequence complementary to a polynucleotide sequence attached to a second particle of the invention. In this way, the blocking strands not only prevent rehybridization of the target polynucleotide but also allow for target polynucleotide recognition by the polynucleotides attached to the first and second particles of the invention.

In still other aspects, the blocking strand may be complementary to the non-target polynucleotide. In these aspects, any number of blocking strands may be used to prevent rehybridization of the non-target polynucleotide to the target polynucleotide as long as the blocking strand does not prevent hybridization of a polynucleotide attached to a first or second nanoparticle of the invention to the target polynucleotide.

In aspects of the methods, only one blocking strand may be used to prevent rehybridization of the target and non-target polynucleotides. In other aspects, two blocking strands may be used, while in still other aspects, three or more blocking strands may be used to prevent rehybridization of the target polynucleotide as long as the blocking strand does not prevent hybridization of a polynucleotide attached to either a first or second nanoparticle of the invention to the target polynucleotide.

In embodiments wherein multiple blocking strands are used, the blocking strands are in one aspect added sequentially or in another aspect, all at once. The order of addition is generally not important as long as the blocking strand does not prevent hybridization of a polynucleotide attached to either a first or second nanoparticle of the invention to the target polynucleotide.

In some aspects wherein the block is a polynucleotide, the length of a blocking strand is about 20 nucleotides. In other aspects, the length of a blocking strand is at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides, or at least 30 nucleotides, or at least 31 nucleotides, or at least 32 nucleotides, or at least 33 nucleotides, or at least 34 nucleotides, or at least 35 nucleotides, or at least 36 nucleotides, or at least 37 nucleotides, or at least 38 nucleotides, or at least 39 nucleotides, or at least 40 nucleotides, or at least 41 nucleotides, or at least 42 nucleotides, or at least 43 nucleotides, or at least 44 nucleotides, or at least 45 nucleotides, or at least 46 nucleotides, or at least 47 nucleotides, or at least 48 nucleotides, or at least 49 nucleotides, or at least 50 nucleotides, or more.

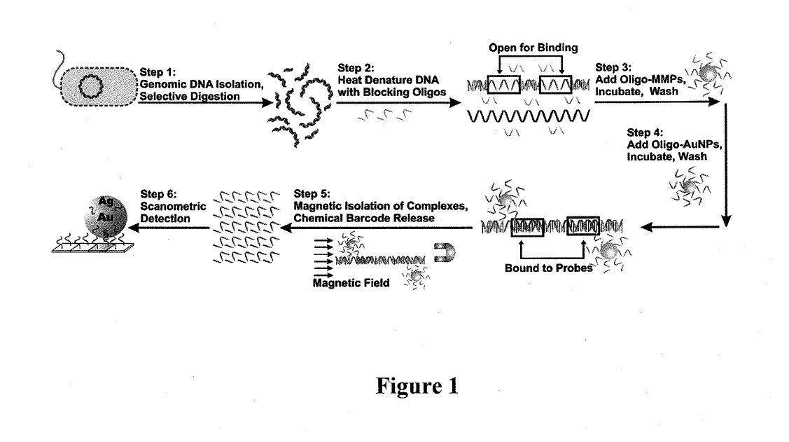

In one embodiment exemplified herein, an assay for detecting the presence of a target polynucleotide is performed by digesting a DNA with a restriction endonuclease to yield smaller DNA fragments. Next, the target DNA is combined with each blocking polynucleotide and mixed and the target strands are denatured. Following denaturation, the samples are then cooled, and oligo-MMPs are added to the reaction vessel which is incubated at conditions sufficient for hybridization to facilitate target capture. Following target capture, the samples are washed, and the oligo-AuNP probes are added to the assay. After hybridization, the particle complexes are washed, and the barcodes chemically released for scanometric detection (Taton et al., Science, 289: 1757-1760 (2000)). This is depicted in FIG. 1. Details of the method are described below.

Nanoparticles useful in the practice of the invention have been described previously (see for example U.S. Pat. No. 6,974,669 and U.S. Pat. No. 7,323,309) and include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods, prisms, or tetrahedra.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99: 14129 (1995); Curtis, A. C., et al., Angew. Chem. Int. Ed Engl., 27: 1530 (1988).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed Engl., 32: 41 (1993); Henglein, Top. Curr. Chem., 143: 113 (1988); Henglein, Chem. Rev., 89: 1861 (1989); Brus, Appl. Phys. A., 53: 465 (1991); Bahncmann, in Photochemical Conversion and Storage of Solar Energy (eds. Pelizetti and Schiavello 1991), page 251; Wang and Herron, J. Phys. Chem., 95: 525 (1991); Olshaysky et al., J. Am. Chem. Soc., 112: 9438 (1990); Ushida et al., J. Phys. Chem., 95: 5382 (1992).

Suitable nanoparticles are also commercially available from, e.g., Ted Pella, Inc. (gold), Amersham Corporation (gold) and Nanoprobes, Inc. (gold).

In one aspect, methods of the invention utilize gold nanoparticles. Gold colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays.

In preparation of nanoparticles useful in the methods, the nanoparticles, the polynucleotides or both are functionalized in order to attach the polynucleotides to the nanoparticles. Such methods are known in the art. For example, polynucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al., Chem. Commun. pages 555-557 (1996) (describes a method of attaching 3′ thiol DNA to flat gold surfaces; this method can be used to attach polynucleotides to nanoparticles). The alkanethiol method can also be used to attach polynucleotides to other metal, semiconductor and magnetic colloids and to the other nanoparticles listed above. Other functional groups for attaching polynucleotides to solid surfaces include phosphorothioate groups (see, e.g., U.S. Pat. No. 5,472,881 for the binding of polynucleotide-phosphorothioates to gold surfaces), substituted alkylsiloxanes (see, e.g., Burwell, Chemical Technology, 4: 370-377 (1974) and Matteucci and Caruthers, J. Am. Chem. Soc., 103: 3185-3191 (1981) for binding of polynucleotides to silica and glass surfaces, and Grabar et al., Anal. Chem., 67: 735-743 (1995) for binding of aminoalkylsiloxanes and for similar binding of mercaptoaklylsiloxanes). Polynucleotides terminated with a 5′ thionucleoside or a 3′ thionucleoside may also be used for attaching polynucleotides to solid surfaces. The following references describe other methods which may be employed to attached polynucleotides to nanoparticles: Nuzzo et al., J. Am. Chem. Soc., 109: 2358 (1987) (disulfides on gold); Allara and Nuzzo, Langmuir, 1: 45 (1985) (carboxylic acids on aluminum); Allara and Tompkins, J. Colloid Interface Sci., 49: 410-421 (1974) (carboxylic acids on copper); Iler, The Chemistry Of Silica, Chapter 6, (Wiley 1979) (carboxylic acids on silica); Timmons and Zisman, J. Phys. Chem., 69: 984-990 (1965) (carboxylic acids on platinum); Soriaga and Hubbard, J. Am. Chem. Soc., 104: 3937 (1982) (aromatic ring compounds on platinum); Hubbard, Acc. Chem. Res., 13: 177 (1980) (sulfolanes, sulfoxides and other functionalized solvents on platinum); Hickman et al., J. Am. Chem. Soc., 111: 7271 (1989) (isonitriles on platinum); Maoz and Sagiv, Langmuir, 3: 1045 (1987) (silanes on silica); Maoz and Sagiv, Langmuir, 3: 1034 (1987) (silanes on silica); Wasserman et al., Langmuir, 5: 1074 (1989) (silanes on silica); Eltekova and Eltekov, Langmuir, 3: 951 (1987) (aromatic carboxylic acids, aldehydes, alcohols and methoxy groups on titanium dioxide and silica); Lec et al., J. Phys. Chem., 92: 2597 (1988) (rigid phosphates on metals).

Any suitable method for attaching polynucleotides onto the nanosphere surface may be used. A particularly preferred method for attaching polynucleotides onto a surface is based on an aging process described in U.S. Pat. Nos. 6,361,944, filed Jun. 25, 1999; 6,506,564, filed Jun. 26, 2000; 6,767,702, filed Jan. 12, 2001; 6,750,016, filed Mar. 28, 2001; U.S. application Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-polynucleotide conjugates with unexpected enhanced stability and selectivity. The method comprises providing polynucleotides preferably having covalently bound thereto a moiety comprising a functional group which can bind to the nanoparticles. The moieties and functional groups are those that allow for binding (i.e., by chemisorption or covalent bonding) of the polynucleotides to nanoparticles. For instance, polynucleotides having an alkanethiol, an alkanedisulfide or a cyclic disulfide covalently bound to their 5′ or 3′ ends can be used to bind the polynucleotides to a variety of nanoparticles, including gold nanoparticles.

General methods for attachment of polynucleotides to nanoparticles to produce stable polynucleotide-nanoparticle conjugates are found in, for example, U.S. Pat. No. 6,974,669.

U.S. Pat. Nos. 6,767,702 and 6,750,016 and international application nos. PCT/US01/01190 and PCT/US01/10071 describe polynucleotides functionalized with a cyclic disulfide which are also contemplated for use in the methods of the invention. The cyclic disulfides preferably have 5 or 6 atoms in their rings, including the two sulfur atoms. Suitable cyclic disulfides are available commercially or may be synthesized by known procedures. The reduced form of the cyclic disulfides can also be used. International application number PCT/US08/063,441 describes polynucleotides functionalized with a triple cyclic disulfide for attachment to silver nanoparticles and is also useful in practicing this invention.

Each nanoparticle will have a plurality of polynucleotides attached to it. As a result, each nanoparticle-polynucleotide conjugate can bind to a plurality of polynucleotides or nucleic acids having the complementary sequence.

Methods of making polynucleotides of a predetermined sequence are well-known. See, e.g., Sambrook et al., Molecular Cloning: A Laboratory Manual (2nd ed. 1989) and F. Eckstein (ed.) Polynucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

In various aspects, methods include polynucleotides which are DNA polynucleotides, RNA polynucleotides, or combinations of the two types. Modified forms of polynucleotides are also contemplated and which include those having at least one modified internucleotide linkage. In one embodiment, the polynucleotide is all or in part a peptide nucleic acid. Other modified internucleoside linkages include at least one phosphorothioate linkage. Additional examples of polynucleotides contemplated for use according to the methods of the invention are generally discussed in International application number PCT/US2006/022325, hereby incorporated by reference in its entirety.

In another aspect of the invention, the barcode polynucleotides released by dehybridization of the particle complex can be detected using a substrate having polynucleotides bound thereto. The polynucleotides have a sequence complementary to at least one portion of the barcode polynucleotides. Some embodiments of the method of detecting the barcode polynucleotides utilize a substrate having complementary polynucleotides bound thereto to capture the barcode polynucleotides. These captured barcode polynucleotides are then detected by any suitable means. By employing a substrate, the detectable change (the signal) can be amplified and the sensitivity of the assay increased.

Any substrate can be used which allows observation of the detectable change. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO)). The substrate can be any shape or thickness, but generally will be flat and thin. Preferred are transparent substrates such as glass (e.g., glass slides) or plastics (e.g., wells of microtiter plates).

Any suitable method for attaching polynucleotides to a substrate may be used. For instance, polynucleotides can be attached to the substrates as described in, e.g., Chrisey et al., Nucleic Acids Res., 24: 3031-3039 (1996); Chrisey et al., Nucleic Acids Res., 24: 3040-3047 (1996); Mucic et al., Chem. Commun., 555 (1996); Zimmermann and Cox, Nucleic Acids Res., 22: 492 (1994); Bottomley et al., J. Vac. Sci. Technol. A, 10: 591 (1992); and Hegner et al., FEBS Lett., 336: 452 (1993).

The polynucleotides attached to the substrate have a sequence complementary to a first portion of the sequence of a barcode polynucleotide to be detected. The barcode polynucleotide is contacted with the substrate under conditions effective to allow hybridization of the polynucleotides on the substrate with the barcode polynucleotide. In this manner the barcode polynucleotide becomes bound to the substrate. Any unbound barcode polynucleotide is preferably washed from the substrate before adding a detection probe such as nanoparticle-polynucleotide conjugates.

In one aspect of the invention, the barcode polynucleotide bound to the polynucleotides on the substrate is contacted with a first type of nanoparticles having polynucleotides attached thereto. The polynucleotides have a sequence complementary to a second portion of the sequence of the barcode polynucleotide, and the contacting takes place under conditions effective to allow hybridization of the polynucleotides on the nanoparticles with the barcode polynucleotide. In this manner the first type of nanoparticles become bound to the substrate. After the nanoparticle-polynucleotide conjugates are bound to the substrate, the substrate is washed to remove any unbound nanoparticle-polynucleotide conjugates.

The polynucleotides on the first type of nanoparticles may all have the same sequence or may have different sequences that hybridize with different portions of the barcode polynucleotide to be detected. When polynucleotides having different sequences are used, each nanoparticle may have all of the different polynucleotides attached to it or, preferably, the different polynucleotides are attached to different nanoparticles. Alternatively, the polynucleotides on each of the first type of nanoparticles may have a plurality of different sequences, at least one of which must hybridize with a portion of the barcode polynucleotide to be detected.

Optionally, the first type of nanoparticle-polynucleotide conjugates bound to the substrate is contacted with a second type of nanoparticles having polynucleotides attached thereto. These polynucleotides have a sequence complementary to at least a portion of the sequence(s) of the polynucleotides attached to the first type of nanoparticles, and the contacting takes place under conditions effective to allow hybridization of the polynucleotides on the first type of nanoparticles with those on the second type of nanoparticles. After the nanoparticles are bound, the substrate is preferably washed to remove any unbound nanoparticle-polynucleotide conjugates.

The combination of hybridizations produces a detectable change. The detectable changes are the same as those described above, except that the multiple hybridizations result in an amplification of the detectable change. In particular, since each of the first type of nanoparticles has multiple polynucleotides (having the same or different sequences) attached to it, each of the first type of nanoparticle-polynucleotide conjugates can hybridize to a plurality of the second type of nanoparticle-polynucleotide conjugates. Also, the first type of nanoparticle-polynucleotide conjugates may be hybridized to more than one portion of the barcode polynucleotide to be detected. The amplification provided by the multiple hybridizations may make the change detectable for the first time or may increase the magnitude of the detectable change. This amplification increases the sensitivity of the assay, allowing for detection of small amounts of barcode polynucleotide.

If desired, additional layers of nanoparticles can be built up by successive additions of the first and second types of nanoparticle-polynucleotide conjugates. In this way, the number of nanoparticles immobilized per molecule of target nucleic acid can be further increased with a corresponding increase in intensity of the signal.

Also, instead of using first and second types of nanoparticle-polynucleotide conjugates designed to hybridize to each other directly, nanoparticles bearing polynucleotides that would serve to bind the nanoparticles together as a consequence of hybridization with binding polynucleotides could be used.

When a substrate is employed, a plurality of the initial types of nanoparticle-polynucleotide conjugates or polynucleotides can be attached to the substrate in an array for detecting multiple portions of a target barcode polynucleotide, for detecting multiple different barcode polynucleotides, or both. For instance, a substrate may be provided with rows of spots, each spot containing a different type of polynucleotide designed to bind to a portion of a target barcode polynucleotide. A sample containing one or more barcode polynucleotides is applied to each spot, and the rest of the assay is performed in one of the ways described above using appropriate polynucleotide-nanoparticle conjugates.

Finally, when a substrate is employed, a detectable change can be produced or further enhanced by silver staining. Silver staining can be employed with any type of nanoparticles that catalyze the reduction of silver. See, International application number PCT/US97/12783, filed Jul. 21, 1997; U.S. Pat. Nos. 6,361,944 and 6,773,884; and Taton et al., Science, 289: 1757-1760 (2000). Preferred are nanoparticles made of noble metals (e.g., gold and silver). See Bassell, et al., J. Cell Biol., 126, 863-876 (1994); Braun-Howland et al. Biotechniques, 13: 928-931 (1992). If the nanoparticles being employed for the detection of a nucleic acid do not catalyze the reduction of silver, then silver ions can be complexed to the nucleic acid to catalyze the reduction. See Braun et al., Nature, 391: 775 (1998). Also, silver stains are known which can react with the phosphate groups on nucleic acids.

Silver staining can be used to produce or enhance a detectable change in any assay performed on a substrate, including those described above. In particular, silver staining has been found to provide a huge increase in sensitivity for assays employing a single type of nanoparticle so that the use of layers of nanoparticles can often be eliminated.

In assays for detecting barcode polynucleotides performed on a substrate, the detectable change can be observed with an optical scanner. Suitable scanners include those used to scan documents into a computer which are capable of operating in the reflective mode (e.g., a flatbed scanner), other devices capable of performing this function or which utilize the same type of optics, any type of greyscale-sensitive measurement device, and standard scanners which have been modified to scan substrates according to the invention (e.g., a flatbed scanner modified to include a holder for the substrate) (to date, it has not been found possible to use scanners operating in the transmissive mode). The resolution of the scanner must be sufficient so that the reaction area on the substrate is larger than a single pixel of the scanner. The scanner can be used with any substrate, provided that the detectable change produced by the assay can be observed against the substrate (e.g., a grey spot, such as that produced by silver staining, can be observed against a white background, but cannot be observed against a grey background). The scanner can be a black-and-white scanner or, preferably, a color scanner. Most preferably, the scanner is a standard color scanner of the type used to scan documents into computers. Such scanners are inexpensive and readily available commercially. For instance, an Epson Expression 636 (600.times.600 dpi), a UMAX Astra 1200 (300.times.300 dpi), or a Microtec 1600 (1600.times.1600 dpi) can be used. The scanner is linked to a computer loaded with software for processing the images obtained by scanning the substrate. The software can be standard software which is readily available commercially, such as Adobe Photoshop 5.2 and Corel Photopaint 8.0. Using the software to calculate greyscale measurements provides a means of quantitating the results of the assays. The software can also provide a color number for colored spots and can generate images (e.g., printouts) of the scans which can be reviewed to provide a qualitative determination of the presence of a nucleic acid, the quantity of a nucleic acid, or both. The computer can be a standard personal computer which is readily available commercially. Thus, the use of a standard scanner linked to a standard computer loaded with standard software can provide a convenient, easy, inexpensive means of detecting and quantitating nucleic acids when the assays are performed on substrates. The scans can also be stored in the computer to maintain a record of the results for further reference or use. Of course, more sophisticated instruments and software can be used, if desired.

### EXAMPLES

### Example 1

Routine growth and maintenance of Bacillus subtilis 168 (American Type Culture Collection #23857) was done in Luria-Bertani (LB) media (LB Broth, Fisher Scientific, BP1427) and on solidified plates using LB agar (LB Agar, Fisher Scientific, BP1425). All cultures were maintained at 30° C. on plates or in liquid form with shaking at 160 rpm and 30° C. All growth media were sterilized by autoclave treatment prior to use.

B. subtilis cells were grown in 50 mLs of liquid media in 125 mL flasks overnight and harvested after 10 hours of growth, generally at an optical density at 600 nm of 2-4 absorbance units/mL. The cells were split into two 25 mL aliquots and spun down at 8,000 rpm for 10 minutes. The supernatant was then removed, and the aliquots were resuspended in 5 mLs of 50 mM Tris, 50 mM EDTA pH 8.0 and frozen for no less than 1 hour at −20° C. Frozen cells in a 25 mL conical tube were placed on ice, and 500 uLs of 10 mg/mL lysozyme (Fisher Scientific) dissolved in 250 mM Tris, pH 8.2 were added to the tube. The cells-lysozyme mixture was allowed to slowly warm to room temperature over a two-hour period. Next, 1 mL of a 1 mg/mL solution of Proteinase K (Fisher Scientific) in 50 mM Tris, 0.4M ETDA, 0.5M SDS, pH 7.5 was incubated with the cells at 50° C. for 1 hour. Afterward, RNase A (1 μL, Ambion Inc./Applied Biosystems) was added to degrade all RNA contamination. Following RNA degradation, the genomic DNA was removed from the other cellular debris by phenol-chloroform extraction and ethanol precipitation. The integrity and size of the genomic DNA was confirmed by gel electrophoresis using a 1% agarose gel with ethidium bromide (Bio-Rad, ReadyAgarose™ Gels) with 1×TBE (Tris Boric Acid, EDTA) buffer at 120 volts for 1 hour. The size of the genomic DNA isolated was compared with commercially available genomic DNA isolated by ATCC for Bacillus subtilis 168.

Probes were designed from the alpha subunit of tryptophan synthase gene (bp 2371552-2370749) from Bacillus subtilis 168. All probes were tested against the NCBI BLAST search engine, with the magnetic and gold probe sequences being unique to B. subtilis. Two of the three blocking sequences (center and 5′) had a homology to one other organism, while the 3′ blocker was specific only for B. subtilis. Probes were designed to fall within a region of the genome that could be cut easily with the restriction enzyme HpyCH4V (New England Biolabs). This was done to allow for de-circularization of the genomic DNA and prevention of super coiling during the heat denaturation step of the assay. Probe specificity was confirmed by routine Southern Blot analysis. Polynucleotide sequences are given in table 1.

TABLE 1

| | | SEQ |
|---|---|---|
| | | ID |
| Name | Sequence | NO |
| Forward Primer | 5′-AGA CTC TAA TGC AGT CAC CAA | 1 |
| | CGC-3′ | |
| Reverse Primer | 5′-TGC TCC CAA TAT AAC GTA TGC | 2 |
| | TGC-3′ | |
| Magnetic Probe | 5′-HS-(CH₂)₆-iSp18-CCG CAA TGA | 3 |
| | GTT CAA TTC ATC CGT GTA CCC-3′ | |
| Gold Probe | 5′-AAG CCA TGA GGT GAC GTA TAT | 4 |
| | TTC TTT AGT-iSp9-AGC TAC GAA | |
| | TAA-(CH₂)₃-SH-3′ | |
| Scanometric | 5′-HS-(CH₂)₆-AAA AAA AAA ATT ATT | 5 |
| | CGT AGC T-3′ | |
| Chip Capture | 5′-ACT AAA GAA ATA TAC GTC ACC | 6 |
| | TCA TGG CTT-(iSp18)₂-NH₂-3′ | |
| CenterBlocking | 5′-TTG AAC AAG CCG AGG GGT TCG | 7 |
| | TCT ACT GTG TAT CT-3′ | |
| 5′ Blocking | 5′-ATT GAC GGT CTG CTT GTT CCG | 8 |
| | GAT CTG CCA TTA GA-3′ | |
| 3′ Blocking | 5′-TGT TCC GGT TGC TGT AGG GTT | 9 |
| | CGG TAT ATC AAA CC-3′ | |

iSpX = Polyethylene Glycol (9 units or 18 Units)

All specialty polynucleotides were purchased from Integrated DNA Technologies and were purified by HPLC. Standard desalting conditions were used for purified PCR primers and blocking strands. Prior to use, the polynucleotides were stored at −80° C. in a dry state. Working solutions of the polynucleotides were stored at −20° C.

The copy number of genomic DNA per milliliter isolated from B. subtilis cells was determined using quantitative real-time PCR (qPCR). A LightCycler 2.0 instrument and LightCycler Software Version 4.0 (Roche Applied Sciences) were used to run the qPCR reactions and quantify the data respectively. Primers were designed to amplify a 1066 base pair (bp) fragment of the genomic DNA from B. subtilis. Primer sequences can be found in Table 1. The LightCycler FastStart DNA Master SYBR Green I kit (Roche Applied Sciences) and the manufacturer's procedure were used to generate the reactions. The reaction was carried out in 20 μL capillaries (Roche Scientific) by placing them in a cooling rack, combining the reagents, spinning the reactants into the capillary and thermally cycling. Because the qPCR products are detected using a fluorescent double stranded DNA binding dye, also referred to as intercalating dyes (e.g., SYBR Green), where total fluorescence depends on product length, a standard curve of the qPCR product had to be synthesized beforehand. To do this, end-point PCR was run using Qiagen's Taq PCR Master Mix following the manufacture's protocol. Within the assay, primer concentrations of 0.5 μM were used with approximately 0.1 μg of template. The reactions were done in 100 μL PCR tubes in strips of eight (Fisher Scientific), on a GeneAmp® PCR System (Applied BioSystems). The thermal profile consisted of 8-minute denaturation step at 95° C., followed by 45 cycles of denaturation at 95° C. for 55 seconds, annealing at 52° C. for 45 seconds, and extension at 72° C. for 120 seconds. Following the 45 cycles, the product was finalized with a 72° C. extension for an additional 10 minutes to allow for all products to be completed. The PCR product size was confirmed by gel electrophoresis using a 1% agarose gel with ethidium bromide (Bio-Rad, ReadyAgarose™ Gels) with 1×TBE (Tris Boric Acid, EDTA) buffer at 120 volts for 1 hour. The remaining PCR product not run on the gel was purified using a MinElute PCR purification kit (Qiagen) following the manufactures instruction. The PCR product was quantified using UV-visible spectroscopy, and run in the qPCR as a standard curve that was used for calibration.

Magnetic Microparticles (MMPs) were prepared according to literature procedures (Hill et al., 2006, Nature Protocols, 1: 324-336). Briefly, 2.8 μM amine functionalized magnetic microparticles (Dynal Corp/Invitrogen) were coupled to thiolated polynucleotide strands using the hetero-bi-functional crosslinker sulfo-SMPB (Pierce Chemical Co). Unreacted amine sites were passivated with sulfo-NHS acetate (Pierce Chemical Co). MMPs were stored at 4° C. in 10 mM phosphate buffered saline (0.15M NaCl) with 0.001% sodium azide as a preservative. MMPs were washed three times prior to use in the assay to remove all storage buffer.

AuNP probes were prepared according to literature procedures (Hill et al., 2006, Nature Protocols, 1: 324-336). Briefly, 4 nanomoles of freshly reduced thiolated DNA was added to 1 mL of 13 nm gold nanoparticles and shaken gently overnight. The system was buffered to a phosphate concentration of 10 mM (pH 7) including 0.01% sodium dodecyl sulfate (SDS). Over the course of one day, the sodium chloride concentration was brought to 0.15 M in a stepwise manner. Particles were then spun (13,000 rpm) and rinsed four times (10 mM phosphate, 0.15 M NaCl, 0.01% SDS, pH 7.4) to remove any unbound DNA. Probes were stored in excess DNA until needed, at which time they were purified as described above.

### Example 2

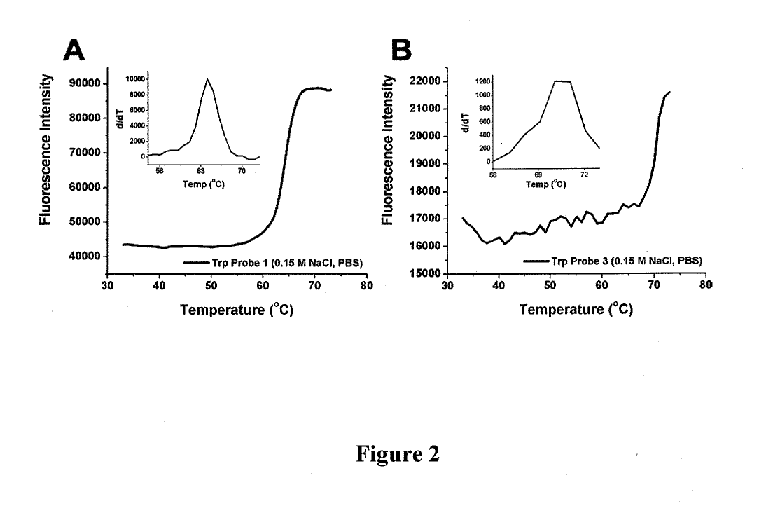

In order to determine the maximum assay stringency, or the upper limit temperature (° C.) to heat denature and eliminate non-specific hybridization events in solution, we determined the melting temperatures (Tₘ) for each of the probes (AuNP and MMP probe sequences) with their respective targets in a 1:1 ratio (FIG. 2). In a typical melting experiment, 13 nm diameter AuNPs were functionalized with a 5′-thiol-modified probe sequence (AuNP probe: 5′-HS-(CH₂)₆-A₁₀-AC TAA AGA AAT ATA CGT CAC CTC ATG GCT T-3′ (SEQ ID NO: 10); magnetic particle probe: 5′-HS-(CH₂)₆-A₁₀-AAA AAA AAA AGG GTA CAC GGA TGA ATT GAA CTC ATT GCG G-3′ (SEQ ID NO: 11)) and allowed to hybridize to one equivalent of a 5′ Fluorescein (FITC)-modified complementary DNA sequence specific to the targeted regions within the tryptophan synthase gene (trp 1, AuNP target): 5′-FITC-AA GCC ATG AGG TGA CGT ATA TTT CTT TAG T-3′ (SEQ ID NO: 12); trp 3, MMP target: 5′-FITC-CC GCA ATG AGT TCA ATT CAT CCG TGT ACC C-3′ (SEQ ID NO: 13)).

All experiments were allowed to equilibrate for over 24 h in 10 mM NaPO₄, 0.15 M NaCl, 0.1% SDS, pH 7.4 (assay buffer) to ensure that equilibrium has been reached. Binding of the nanoparticle probes to a complementary target sequence modified with a molecular fluorophore resulted in quenching and decreased fluorescence intensity (FIG. 2). Subsequent heating resulted in dissociation of the probe/target complex and an increase in fluorescence intensity, providing a way to spectroscopically monitor the melting transition (FIG. 2). Fluorescence measurements were performed on a Molecular Devices Gemini EM Microplate spectrofluorometer with temperature control. Comparison of the trp 1 probe-target (Tₘ=64.1±0.5° C.) and trp 3 probe-target (Tₘ=70.1±1.1° C.) complexes after dissociation revealed a difference of 6° C. in the Tₘ. Using the lowest Tₘ value the appropriate thermal stringency can be applied in the bio-barcode assay to achieve specific nanoparticle probe-target hybridization.

### Example 3

Bacillus subtilis cells were isolated from culture by centrifugation, and the genomic DNA was isolated as described above using lysozyme and proteinase K. The genomic DNA was then cut using the restriction endonuclease HpyCH4 V (New England Biolabs, R0620L). A restriction digestion step was needed to prevent super-coiling during heating and subsequent detection. A dilution series of the unknown concentration genomic DNA was made for testing with the bio-barcode assay. Additionally, an aliquot of the genomic DNA was quantified using qPCR as described above. Assays were assembled in nuclease-free eppendorf tubes (Ambion Inc) containing 5 μLs of unknown genomic DNA sample, 1 μL of each blocking polynucleotide (200 μM stocks) and 32 μLs of assay buffer (10 mM PO4, 0.15 M NaCl, 0.1% SDS, pH 7.4). The assays were mixed thoroughly and placed at 95° C. for 10 minutes to denature the genomic DNA fragments. After 10 minutes the temperature was lowered to 72° C., and 10 μLs of MMPs (20 mg/mL) were added to each well. The reactions were mixed well, and placed at 40° C. for 2 hours while being mixed in an end over end manner to ensure that the MMPs did not settle. The MMPs with the target bound were then washed 3 times with 100 μLs of assay buffer to remove all unbound nucleic acids and remaining components for the restriction digest, especially dithiothreitol (DTT), which can react with the AuNP probes in the next step. To the washed MMPs 40 μLs of assay buffer and 10 μLs of 500 pM freshly cleaned AuNPs were added. The detections were vortexted, and placed at 37° C. with end-over-end mixing for one hour. The reactions were then washed five times using 1000 μLs of assay buffer to remove all unbound AuNPs. The supernatant was removed after the 5th wash and the complexes were resuspended in 50 μLs of 0.5M DTT in assay buffer, and placed at 50° C. for 15 minutes, and 45 minutes 25° C. under vortex. The DTT solution liberates the thiolated polynucleotide barcodes from the surface of the gold nanoparticle through ligand exchange. Following barcode release, the MMPs are isolated using a magnet and 15 μLs of each sample was added to a different well on the chip. The barcode samples were heated to 60° C. and then allowed to hybridize to the chip for 1 hour at 37° C. while shaking at 120 rpm. The chips were then washed three times in 1×PBS and reassembled with clean gaskets. To each well 15 μLs of universal probe solution (500 pM universal AuNP, 10% formamide in assay buffer) was added. The probes were allowed to hybridize for 45 minutes at 37° C. while shaking at 120 rpm. The gaskets were then disassembled, and the slides washed three times in 0.5 M NaNO₃, 0.2% Tween 20, 0.1% SDS and washed twice in 0.5 M NaNO₃ and finally quickly dipped in cold (4° C.) 0.1 M NaNO₃. The slides were spun dry, and equal parts silver stain solution A and B (Nanosphere Inc) were placed on top of the slide so that the entire surface was covered. The silver enhancement was carried out for 3 minutes before being terminated by washing with nanopure water. The slide was re-dried and imaged using a high resolution VERIGENE ID (Nanosphere Inc). The spot intensity was analyzed using GenePix Software (Molecular Devices).

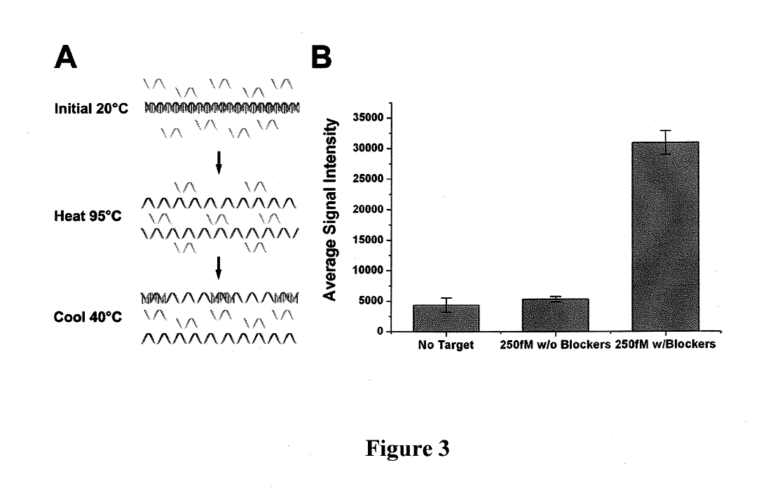

To detect genomic DNA using the bio-barcode assay, separation of the duplex stands into their single strand forms is critical to allow probe binding. However, the conditions required to thermally denature DNA are very harsh (95° C.), and the oligo-MMPs (iron oxide nanoparticles embedded in a polymer scaffold) deteriorate under such stresses. Still, chemical denaturants are not an option, as they would prevent the oligo-MMPs from hybridizing to the target as well. To overcome the challenge of denaturing DNA duplexes and keeping them apart long enough to allow the oligo-MMPs to hybridize required the implementation of blocking polynucleotides. These blocking polynucleotides consisted of three different 35 base pair sequences, designed to flank the particle probe binding sites. In the assay, the blockers were used in great excess (1:106, target: blocker) to prevent strand rehybridization (Minunni et al., Am. Chem. Soc., 127: 7966-7967 (2005)). As can be seen in the scheme shown in FIG. 3A, when the duplex DNA is heated with an excess of blockers, the duplex thermally denatures, and as the solution cools, the kinetics of blocker binding should be faster than that of the native strand re-hybridizing (Minunni et al., Analytica Chimica Acta 526: 19-25 (2004). This should result in open regions of the duplex. The data presented in FIG. 3B shows the bio-barcode assay run under various conditions to test the effectiveness of the blockers. The left most sample was run with digested λ-phage DNA as a negative control and 4 μM of each of the three blocking polynucleotides, the sample in the center was run with 250 fM target only, and the third sample was run with 250 fM target and 4 μM of each of the three blockers. The impact of the blockers is astounding. The signals obtained for the λ-phage DNA and the target without blocking strands fall within each other's standard deviations, while the target sample that contained the blockers shows a six-fold increase in signal.

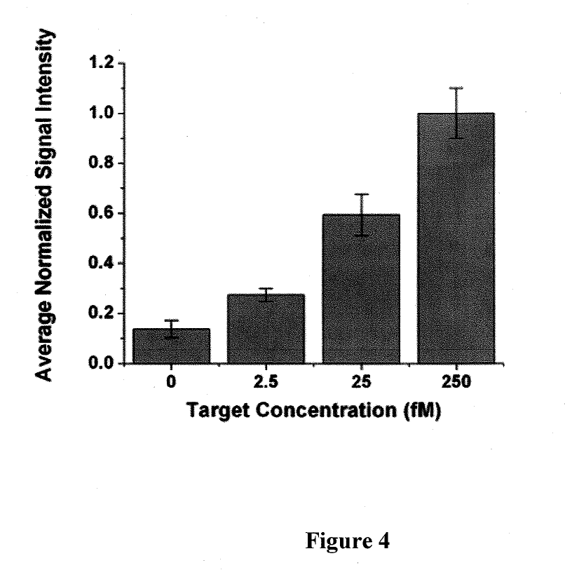

In order to evaluate the sensitivity of the assay in the presence of blockers, a digestion mixture was diluted into a series differing in concentration by orders of magnitude. The data presented in FIG. 4 shows that the bio-barcode assay is capable of detecting bacterial genomic DNA down to the low femtomolar concentration range, with a final sensitivity of 2.5 fM (final concentration in the assay, 7.5×10⁴ copies). Here, N-hydroxy succinimide (NHS) activated CODELINK glass microscope slides (GE-Healthcare) were used to support microarrays of amine-terminated polynucleotides complementary to the particle-bound barcode sequences according to the manufacturer's protocol. The “capture” polynucleotides were printed in triplicate using a GME 418 robotic pin-and-ring microarrayer (Affymetrix). The chips were allowed the react overnight at 70% humidity, and were then passivated in 0.2% SDS as 50° C. for 30 minutes to hydrolyze all remaining NHS groups. A slide presented in false color was produced where red and white indicate high signal intensity, and blue and black indicate low signal intensity. A row of spots labeled 250 fM was the most intense red color, indicating the strongest signal. The spots at 25 fM target concentration showed an orange/yellow color indicating a moderate to high signal intensity. The 2.5 fM spots showed a yellow/green intensity, which is distinct from the blue color seen at the 0 fM (no target) row at the top of the slide. Additionally, the quantified data (5 independent experiments) presented in FIG. 4 shows that the signal at 2.5 fM (0.273) is greater than three standard deviations (3*0.0258=0.0774) above the one-standard deviation value for the control (0.138+0.0353=0.1732), (0.1956>0.1732). The normalized assay is log linear through the femtomolar concentration range, and becomes non-linear above 1 pM due to saturation of the scattering signal as read by the Verigene ID (Nanosphere Inc). This saturation issue can be easily solved by diluting the barcodes prior to their detection by the scanometric method therefore allowing one to tailor the dynamic range of the bio-barcode assay for their expected concentration range (Taton et al., Science, 289: 1757-1760 (2000)).

What is claimed:

1. A method for detecting presence of a target polynucleotide in a sample comprising the step of:
detecting said target polynucleotide in a particle complex, components of said particle complex comprising:
(i) said target polynucleotide;
(ii) a first particle having a first polynucleotide attached thereto, wherein all or part of said first polynucleotide is specifically hybridized to a first binding complement in said target polynucleotide;
(iii) a second particle having a second polynucleotide attached thereto and a DNA barcode hybridized to a first site in said second polynucleotide, wherein said second polynucleotide is specifically hybridized to a second binding complement in said target polynucleotide through a second site in said second polynucleotide; and
(iv) a blocking polynucleotide hybridized to a third binding complement in said target polynucleotide, wherein hybridization of said blocking polynucleotide to said target polynucleotide prevents said target polynucleotide from hybridizing to its complementary sequence,
wherein the particle complex is in an environment that promotes dehybridization of said DNA barcode from said complex, the detection of said DNA barcode indicating the presence of said target polynucleotide.

2. The method of claim 1 wherein said particle complex is isolated prior to dehybridization of said DNA barcode.

3. The method of claim 1 wherein said particle complex is formed by sequential addition of one or more solutions of components which form said particle complex to a solution containing said target polynucleotide.

4. The method of claim 1 wherein said particle complex is formed by sequential addition of a solution containing said target polynucleotide to one or more solutions of components which form said particle complex.

5. The method of claim 1 wherein said first particle is magnetic.

6. The method of claim 5 wherein said particle complex is isolated using a magnet prior to dehybridization of said DNA barcode.

7. The method of claim 1 wherein said second particle is a nanoparticle.

8. The method of claim 7 wherein said nanoparticle is a metallic nanoparticle.

9. The method of claim 8 wherein said metallic nanoparticle is a gold nanoparticle.

10. The method of claim 1 wherein said target polynucleotide is a naturally occurring polynucleotide.

11. The method of claim 1 wherein said target polynucleotide is a synthetic polynucleotide.

12. The method of claim 11 wherein said synthetic polynucleotide is a peptide nucleic acid.

13. The method of claim 1 wherein the target polynucleotide is a polynucleotide that forms intermolcular or intramolecular double-stranded structure that precludes particle complex formation.

14. The method of claim 13 wherein said molecule is selected from the group consisting of DNA or RNA.

15. The method of claim 1 wherein said target polynucleotide is a bacterial polynucleotide.

16. The method of claim 15 wherein said target polynucleotide is bacterial genomic DNA.

17. The method of claim 1 wherein said target polynucleotide is a viral polynucleotide.

18. The method of claim 17 wherein said viral polynucleotide is viral genomic DNA.

19. The method of claim 1 wherein said polynucleotide is a fungal polynucleotide.

20. The method of claim 19 wherein said fungal polynucleotide is fungal genomic DNA.

21. The method of claim 1 further comprising the steps of:
(a) denaturing a target polynucleotide having a double stranded polynucleotide region;
(b) hybridizing said target polynucleotide to said blocking polynucleotide;
(c) hybridizing said target polynucleotide to said first polynucleotide bound to said first particle;
(d) washing said target polynucleotide to remove any first polynucleotide on said first particle that is not hybridized to said target polynucleotide;
(e) hybridizing said target polynucleotide to said second polynucleotide bound to said second particle;
(f) washing said target polynucleotide to remove any second polynucleotide on said second particle that is not hybridized to said target polynucleotide;
(g) isolating said particle complex comprising said target polynucleotide having said blocking polynucleotide hybridized thereto, said first polynucleotide on said first particle hybridized thereto, and said second polynucleotide on said second particle hybridized thereto;
(h) dehybridizing said DNA barcode from said second polynucleotide bound to said second particle; and
(i) detecting said DNA barcode, thereby indicating presence of said target polynucleotide.


SEQUENCE LISTING (13 sequences)

SEQ ID NO: 1 - DNA, 24 bp, Artificial Sequence
  random synthetic oligonucleotide sequence
agactctaatgcagtcaccaacgc

SEQ ID NO: 2 - DNA, 24 bp, Artificial Sequence
  random synthetic oligonucleotide sequence
tgctcccaatataacgtatgctgc

SEQ ID NO: 3 - DNA, 30 bp, Artificial Sequence
  random synthetic oligonucleotide sequence
ccgcaatgagttcaattcatccgtgtaccc

SEQ ID NO: 4 - DNA, 43 bp, Artificial Sequence
  random synthetic oligonucleotide sequence
aagccatgaggtgacgtatatttctttagtnagctacgaataa

SEQ ID NO: 5 - DNA, 22 bp, Artificial Sequence
  random synthetic oligonucleotide sequence
aaaaaaaaaattattcgtagct

SEQ ID NO: 6 - DNA, 30 bp, Artificial Sequence
  random synthetic oligonucleotide sequence
actaaagaaatatacgtcacctcatggctt

SEQ ID NO: 7 - DNA, 35 bp, Artificial Sequence
  random synthetic oligonucleotide sequence
ttgaacaagccgaggggttcgtctactgtgtatct

SEQ ID NO: 8 - DNA, 35 bp, Artificial Sequence
  random synthetic oligonucleotide sequence
attgacggtctgcttgttccggatctgccattaga

SEQ ID NO: 9 - DNA, 35 bp, Artificial Sequence
  random synthetic oligonucleotide sequence
tgttccggttgctgtagggttcggtatatcaaacc

SEQ ID NO: 10 - DNA, 40 bp, Artificial Sequence
  random synthetic oligonucleotide sequence
aaaaaaaaaaactaaagaaatatacgtcacctcatggctt

SEQ ID NO: 11 - DNA, 50 bp, Artificial Sequence
  random synthetic oligonucleotide sequence
aaaaaaaaaaaaaaaaaaaagggtacacggatgaattgaactcattgcgg

SEQ ID NO: 12 - DNA, 30 bp, Artificial Sequence
  random synthetic oligonucleotide sequence
aagccatgaggtgacgtatatttctttagt

SEQ ID NO: 13 - DNA, 30 bp, Artificial Sequence; identical to SEQ ID NO: 3
  random synthetic oligonucleotide sequence